(19) United States
(12) Patent Application Publication
(10) Pub. No.: US 2010/0249620 A1
(43) Pub. Date: Sep. 30, 2010

(54) USE OF BLOOD FLOW PARAMETERS TO DETERMINE THE PROPENSITY FOR ATHEROTHROMBOSIS

(76) Inventor: Daniel J. Cho, Chesterbrook, PA (US)

Correspondence Address: OSTROLENK FABER GERB & SOFFEN, 1180 AVENUE OF THE AMERICAS, NEW YORK, NY, 100368403, US

(21) Appl. No.: 12/668,270

(22) PCT Filed: Jul. 11, 2008
(86) PCT No.: PCT/US08/08551

§ 371 Date: Jun. 14, 2010

Related U.S. Application Data
(60) Provisional application No. 60/949,008, filed on Jul. 11, 2007.

(51) Int. Cl. A61B 5/026; G06F 19/00

(52) U.S. Cl. 600/504; 702/19

(57) ABSTRACT

A method for determination of the risk of atherothrombosis that includes determining blood shear stress based on blood viscosity and comparing the blood shear stress to a critical threshold blood shear stress indicative of the propensity of plaque to rupture.

### RELATED APPLICATION

This application is based on and claims priority to U.S. Provisional Application No. 60/949,008, filed Jul. 11, 2007, entitled Use of Blood Flow Parameters to Determine the Propensity for Atherothrombosis to which a claim of priority is hereby made and the disclosure of which is incorporated by reference.

### FIELD OF THE INVENTION

The present invention relates generally to the use of blood flow parameters such as whole blood viscosity, shear stress, and shear rate to measure and determine the propensity for atherothrombosis—the tendency of atherosclerotic plaque to rupture or the vulnerability of atherosclerotic plaque—for the purpose of preventing and/or treating acute coronary syndromes.

### REFERENCES

The following references are referred to below using its assigned number. The disclosure of each reference is incorporated by reference.
1. Dirksen M T, van der Wal A C, van der Berg F M, van der Loos C M, Becker A E, Distribution of inflammatory cells in atherosclerotic plaques relates to the direction of flow, Circulation, 1998; 98: 2000-2003.
2. Davies M J, Richardson P D, Woolf N, Katz D R, Mann J. Risk of thrombosis in human atherosclerotic plaques: role of extracellular lipid, macrophage, and smooth muscle cell content. Br Heart J. 1993; 69: 377-381.
3. Gown A M, Tsukada T, Ross R. Human atherosclerosis, II: immunocytochemical analysis of the cellular composition of human atherosclerotic lesions. Am J Pathol. 1986; 125: 191-207.
4. van der Wal A C, Becker A E, van der Loos C M, Tigges A J, Das P K. Fibrous and lipid-rich plaques are part of interchangeable morphologies related to inflammation: a concept. Coron Artery Dis. 1994; 5: 463-469.
5. Moreno P R, Falk E, Palacios J F, Newell J B, Fuster V, Fallon J T. Macrophage infiltration in acute coronary syndromes: implications for plaque rupture. Circulation. 1994; 90: 775-778.
6. van der Wal A C, Becker A E, van der Loos C M, Das P K. Site of intimal rupture or erosion of thrombosed coronary atherosclerotic plaques is characterized by an inflammatory process irrespective of the dominant plaque morphology. Circulation. 1994; 89: 36-44.
7. Richardson P D, Davies M J, Born G V R. Influence of plaque configuration and stress distribution on fissuring of coronary atherosclerotic plaques. Lancet. 1989; 2: 941-944.
8. Shah P K, Falk E, Badimon J J, Fernandez-Ortiz A, Mailhac A, Villareal-Levy G, Fallon J T, Fuster V. Human monocyte-derived macrophages induce collagen breakdown in fibrous caps of atherosclerotic plaques. Circulation. 1995; 92: 1565-1569.
9. Lendon C L, Davies M J, Born G V R, Richardson P D. Atherosclerotic plaque caps are locally weakened when macrophages density is increased. Atherosclerosis. 1991; 87: 87-90.
10. Davies M J. A macro and microview of coronary vascular insult in ischaemic heart disease. Circulation. 1990; 82(suppl. II): II-38-II-46.
11. van der Wal A C, Becker A E, Koch K T, Piek J J, Teeling P, van der Loos C M, David G K. Clinically stable angina pectoris is not necessarily associated with histologically stable atherosclerotic plaques. Heart. 1996; 76: 312-316.
12. Zarins C K, Bomberger R A, Glagov S. Local effects of stenoses: increased flow velocity inhibits atherogenesis. Circulation. 1981; 64(suppl. II): II-221-II-227.
13. Levesque M J, Liepsch D, Moravec S, Nerem R M. Correlation of endothelial cell shape and wall shear stress in a stenosed dog aorta. Arteriosclerosis. 1986; 6: 220-229.
14. Slager C J, Wentzel J J, Gijsen F J H, Thury A, van der Wal A C, Schaar J A and Serruys P W. The role of shear stress in the destabilization of vulnerable plaques and related therapeutic implications, Nature Clin Prac Cardiovasc Med. 2005; 2: 456-464.
15. Libby P. Molecular bases of the acute coronary syndrome, Circulation. 1995; 91: 2844-2850.
16. Gertz S D, Roberts W C. Hemodynamic shear stress in rupture of coronary arterial atherosclerostic plaques, Am J Cardiol. 1990; 66: 1368-1372.
17. Chapman I. Morphogenesis of occluding artery thrombosis. Arch Pathol Lab Med. 1965; 80: 256-261.
18. Friedman M, Van Den Bovenkamp G J. The pathogenesis of a coronary thrombus. Am J Pathol. 1966; 48: 19-31.
19. Ridolfi R L, Hutchins G M. The relationship between coronary artery lesions and myocardial infarcts: ulceration of atherosclerotic plaques precipitating coronary thrombosis. Am Heart J. 1977; 93: 468-486.
20. Horie T, Sekiguchi M, Hirosawa K. Coronary thrombosis in pathogenesis of acute myocardial infarction: histopathological study of coronary arteries in 108 necropsied casts using serial section. Br Heart J. 1978; 40: 153-161.
21. Falk E. Plaque rupture with severe pre-existing stenosis precipitating coronary thrombosis: characteristics of coronary atherosclerotic plaques underlying fatal occlusive thrombi. Br Heart J. 1983; 50: 127-134.
22. Ambrose J A, Winters S L, Stern A, Eng A, Teichholz L E, Gorlin R, Fuster V. Angiographic morphology and the pathogenesis of unstable angina pectoris. J Am Coll Cardiol. 1985; 5: 609-616.
23. Fuster V, Badimon L, Cohen M, Ambrose J A, Badimon J J, Chesebro J. Insights into the pathogenesis of acute ischemic syndromes. Circulation. 1988; 77: 1213-1220.
24. Davies M J. Successful and unsuccessful coronary thrombolysis. Br Heart J. 1989; 61: 381-384.
25. Falk E. Morphologic features of unstable atherothrombotic plaques underlying acute coronary syndromes. Am J Cardiol. 1989; 63: 114E-120E.
26. Molter J E, Tofler G H, Stone P H. Circadian variation and triggers of onset of acute cardiovascular disease. Circulation. 1989; 79: 733-743
27. Alpert J S. The pathophysiology of acute myocardial infarction. Cardiology. 1989; 76: 85-95.
28. Gertz S D, Uretzky G, Wajnberg R S, Navot N, Gotsman M S. Endothelial cell damage and thrombus formation after partial arterial constriction: relevance to the role of coronary artery spasm in the pathogenesis of myocardial infarction. Circulation. 1981; 63: 416-486.
29. Nelson E, Gertz S D, Forbes M S, Rennels M I, Heald F P, Kahn M A, Farber T M, Miller. E, Husain M M, Earl F L. Endothelial lesions in the aorta of egg yolk-fed miniature swine: a study by scanning and transmission electron microscopy. Exp Mel Pathol. 1976; 25: 208-220.
30. Flaherty J T, Pierce J E, Ferrans V J, Patel D J, Tucker W K, Fry D L. Endothelial nuclear patterns in the canine arterial tree with particular reference to hemodynamic events. Circ Res. 1972; 30: 23-33.
31. Wright H P. Endothelial turnover. In: Koller F, Brinkhous K M, Biggs R, Rodman N F, Hinnom S, eds. Vascular Factors and Thrombosis. Stuttgart: FK Schattauer Verlag, 1970: 79-84.
32. Fry D L. Response of arterial wall to certain physical factors. In: Porter R, Knight J, eds. Atherogenesis: Initiating Factors. Ciba Foundation Symposium 12. Amsterdam: Ciba Foundation, 1973: 93-125.
33. Fry D L. Hemodynamic forces in atherogenesis. In: Scheinberg P, ed. Cerebrovascular Diseases. New York: Raven Press, 1976: 77-95.
34. Fry D L. Acute vascular endothelial changes associated with increased blood velocity gradients. Circ Res. 1968; 22: 165-197.
35. Back L H, Radbill J R, Cho Y I, Crawford D W. Measurement and Prediction of Flow through an Axisymmetric Replica of a Mildly Atherosclerotic Coronary Artery of Man. J Biomech. 1986; 19: 1-17.
36. Fox R W, McDonald A T, Pritchard P J. Introduction to Fluid Mechanics. New York: Wiley, 2006.

### DEFINITIONS

Systolic blood viscosity refers to blood viscosity at high shear rates (shear rates equal to or higher than 50 s⁻¹, e.g., at 300 s⁻¹ or higher). Diastolic blood viscosity refers to blood viscosity at low shear rates (shear rates at or below 25 s⁻¹, e.g., at 1 s⁻¹ or lower). Similarly, systolic shear stress refers to wall shear stress in the high shear rate flow regime (shear rates equal to or higher than 50 s⁻¹, e.g., at 300 s⁻¹ or higher). Diastolic shear stress refers to wall shear stress in the low shear rate flow regime (shear rates at or below 25 s⁻¹, e.g., at 1 s⁻¹ or lower). These definitions are useful for characterizing the behavior of blood flow and are important to the clinical application of blood flow parameters.

### Relevant Art

Dirksen, et al. have reported that atherosclerotic plaques show marked variability with respect to the distribution of inflammatory cells, not only from one lesion to another lesion but also within one and the same plaque [1-4]. They considered this marked variability to be significant because plaque inflammation is widely considered to play a role in plaque destabilization and, eventually, plaque erosion and rupture [1, 5-9]. It has been observed that plaques dominated by smooth muscle cells (SMCs) are considered stable. Indeed, coronary atherectomy specimens obtained from patients with chronic stable angina contained SMCs as the dominant cellular component, but in those obtained from patients with unstable angina or acute myocardial infarction, inflammation prevailed [5, 10, 11].

The mechanism responsible for these large variations in the cellular composition of atherosclerotic plaques is not yet fully understood. Dirksen, et al. [1] have indicated that the geometry of a bulging plaque dictates differences in the impact of blood flow in relation to the direction of flow. Published research has shown that the luminal endothelial lining on the upstream (proximal) sites of plaque is under elevated systolic shear stress, whereas at the downstream (distal) sites, elevated diastolic shear stress prevails [12, 13]. Dirksen and his collaborators [1] speculated that differences in wall shear stresses also have implications for local variation in the cellular composition of plaques.

The distribution of macrophages and smooth muscle cells (SMCs) within atherosclerotic plaques is highly variable, which is important clinically because these differing cell types affect the stability of atherosclerotic plaques. Dirksen, et al. [1] investigated whether local variations in arterial flow over the plaque surface could relate to differences in the distribution of SMCs and macrophages in plaques. They collected 33 carotid plaques, compared the cell composition of upstream parts (where high shear rates and systolic flow conditions prevail) with that of downstream parts (where low shear rates and diastolic flow conditions prevail) and concluded that: seventy percent of plaques showed more SMCs in their downstream part, and 67% of plaques contained more macrophages in the upstream part; immunostained macrophage areas were larger in the upstream parts; immunostained SMC areas were larger in downstream parts; and rupture sites of 6 of 9 ruptured plaques were in the upstream part. Thus, they concluded that there are significant differences in cell composition between upstream and downstream parts of plaques, and that such differences may explain why plaques rupture upstream more often. That is, Dirkson, et al. have posited that plaque composition may be indicative of its propensity to rupture.

Slager, et al. published a review article [14] reporting that type-IV plaques (AHA classification), consisting of a lipid core covered by a fibrous cap, tend to develop at locations of eccentric low shear stress. Shear stress was not actually used therein to identify the location of growth; rather, Slager, et al., discussed possible mechanisms, including shear stresses, that could explain otherwise paradoxical observations and outcomes. For instance, they indicated that vascular remodeling initially preserves the lumen diameter while maintaining the low shear stress conditions that encourage plaque growth.

Generally cardiologists tend to define vulnerable plaques as thin-cap, rupture-prone fibroatheromas. Slager, et al. [14] noted that the rupture of thin-cap fibroatheromas is the most common cause of arterial thrombotic events, or atherothrombosis, and occurs most frequently at the entrance section of stenoses of low severity. Shear stress induces important biological effects in endothelial cells (ECs) that can affect the crucial balance between cap-reinforcing matrix synthesis by synthetic smooth-muscle cells (SMCs) and matrix breakdown by metalloproteinases, which are produced by macrophages [6, 15].

Gertz and Roberts also studied the role of hemodynamic shear force on coronary arterial atherosclerotic plaques [16]. They reported that several angiographic and necropsy studies suggested a direct pathogenetic relation among ruptures of a coronary arterial atherosclerotic plaque, coronary thrombus formation, and acute myocardial infarction (AMI) [17-27]. However, they pointed out that the local pathophysiologic factor or factors responsible for the initiation of plaque rupture have not been identified, although there have been some suggestions, including hemorrhage into a plaque after injury to vasa vasora; mechanical compression associated with coronary spasm; increased intraluminal arterial pressure; and circumferential tensile stress on the “fibrous cap” of the plaque. They do, also, present shear force—or shear stress—as a factor in the pathogenesis of plaque rupture.

Using correlative scanning electron microscopic and blood flow studies of the coronary arteries of dogs and the common carotid artery of rabbits, Gertz and his colleagues [28] showed that marked endothelial damage, with extensive platelet deposition and thrombus formation on exposed subendothelial tissues, occur at the site of a partial arterial constriction (40 to 60% reduction in transluminal diameter), and perhaps particularly, when the reduction in luminal diameter is insufficient to alter substantially the rate of distal coronary blood flow. This observation is supported by reports of functional and structural changes in the endothelial lining [29-33] associated with arterial curvatures and “flow dividers” of branch orifices.

### SUMMARY OF THE INVENTION

A method according to the present invention is directed at determining the risk of rupture of a portion of a plaque formation residing in a lumen of a human blood vessel by calculating a reference blood viscosity value based on a reference blood shear rate value, calculating a reference blood shear stress value based on the reference blood viscosity value, comparing the reference blood shear stress value to a critical threshold shear stress value indicative of a critical shear stress required to rupture plaque to determine whether the blood shear stress value has at least reached the critical shear stress value. Preferably, a risk ratio can be determined based on the result of the comparison between the reference blood shear stress value and the critical threshold shear stress value.

In the first embodiment, the reference blood shear rate may be a value that is higher than 50 s⁻¹ and preferably higher than 300 s⁻¹ resulting in a systolic blood viscosity value which is then used as a reference blood viscosity value.

In the second embodiment, the reference blood shear rate may be a value that is lower than 25 s⁻¹ and preferably lower than 1 s⁻¹ resulting in a diastolic blood viscosity value which is then used as a reference blood viscosity value.

In the third embodiment, the reference blood shear stress value is calculated based on the reference blood viscosity value and the percent blockage of the lumen by the plaque formation. Furthermore, an increased blood flow velocity value, due to, for example, exercise, can be used to determine reference shear stress at elevated blood flow rates.

In the fourth embodiment, the reference blood shear stress value is calculated based on the reference blood viscosity value and the diameter of the lumen at a specific location corresponding to a location on the plaque formation. In this embodiment, the diameter of the lumen is obtained by first obtaining a profile of the plaque formation in the lumen. The profile of the plaque in the lumen can be obtained through angiography, interferometric phase-contrast imaging, magnetic resonance imaging, three-dimensional MR angiography, computed tomography (CT), intravascular ultrasound, virtual arterial endoscopy, or endovascular probe. In the fourth embodiment, the reference blood shear rate may depend on the location that is selected for reference shear stress calculation. Thus, if the location is a high shear rate location, a systolic blood viscosity based on a blood shear rate higher than 50 s⁻¹ or higher than 300 s⁻¹ may be used. If the location is a low shear rate location, a diastolic blood viscosity based on a blood shear rate lower than 25 s⁻¹ or lower than 1 s⁻¹ is used.

### BRIEF DESCRIPTION OF THE DRAWINGS

FIG. 1 illustrates a plaque formation inside an artery.

FIG. 2 graphically illustrates a relationship between wall shear stress for a number of blood viscosity values at rest as a function of percent blockage of lumen.

FIG. 3 graphically illustrates a relationship between wall shear stress for a number of blood viscosity values during exercise as a function of blockage of lumen.

FIG. 4 graphically illustrates threshold shear stress value for a number of blood viscosity values as a function of percent blockage of lumen.

FIG. 5 shows the results of a study reporting wall shear stress changes along a surface of a plaque formation.

FIG. 6 shows a plot of values indicating possible peak shear stress values at the maximum percent blockage as a function of flow rate for different Reynolds numbers.

### DETAILED DESCRIPTION OF EMBODIMENTS OF THE INVENTION

The present invention relates a method of using blood viscosity and shear stress measurements to assess the tendency of atherosclerotic plaque (hereafter plaque) to rupture.

The work of Dirksen et al. suggests a role for arterial flow in the distribution of different cell types. The low shear regime areas at the downstream shoulders of plaques contain significantly more SMCs, which provide the background stimuli for progressive growth at distal ends of plaques. On the other hand, the significantly higher number of macrophages on the upstream shoulders of plaques suggests a connection between high shear stress conditions and plaque instability.

Hemodynamic factors, such as wall shear stress, have been hypothesized to play a role in plaque growth as well as in plaque stability, but no method has been proposed by which the likelihood of plaque rupture is quantifiably assessed; nor has any methodology been proposed for diagnostic assessment of the tendency for plaque rupture using hemodynamic (or blood flow) parameters.

The present application discloses a method by which blood viscosity and shear stress are used, individually as quantitative thresholds, as well as together, and additionally, in conjunction with other parameters in a diagnostic assessment framework to determine the risk of plaque rupture.

Fry [34] reported his observations of an animal study where a critical wall shear stress value of 379±85 dyne/cm² was found to damage endothelial cells. These observations involved the aorta of dogs and were made at peak systole (i.e. high shear rate) conditions assuming steady, laminar flow. Moreover, the observations involved endothelial cells lining the inner wall of canine aorta and not actual atherosclerotic plaques. As Gertz and Roberts noted in their review editorial [16], although Fry's study did not directly relate to the rupture of plaque, it does suggest that wall shear stress may play a role in affecting the cellular structure at the inner surface of a vessel such as an artery. More particularly, because it has been shown that endothelial tissue can be damaged at a critical shear stress value, the present application presents a way to utilize hemodynamic parameters to quantify the tendency of plaque to rupture.

A threshold wall shear stress, i.e., a wall shear stress value beyond which plaque is expected to rupture, can be obtained through a clinical outcomes trial having a primary endpoint of major arterial thrombotic events. By utilizing the diagnostic parameters of blood viscosity, lumen diameter using vessel imaging, and wall shear stress, critical threshold values can be obtained. One example of the statistical analysis of clinical endpoints is as follows: Study subjects would be grouped based on their maximum shear stress values (e.g., two groups comprised of high versus low values, tertiles, quartiles, etc.). By comparing the number of arterial thrombotic events in the groups, a risk ratio can be calculated, i.e. the increased risk of event for one group with respect to another. Confidence intervals and p-values can be calculated to test reliability and significance.

In addition to empirical studies, the tendency for atherothrombosis can be determined analytically by taking into account the mechanical properties of a plaque formation, including, for example, the cellular composition thereof. This embodiment of the present application incorporates hemodynamic parameters such as shear stress into a diagnostic framework using, for example, finite element analysis, together with vessel imaging and analysis of the biochemical content of the blood. Vessel imaging techniques are wide-ranging and include ultrasonic methods, x-ray, magnetic resonance imaging, computed tomography, among others. For the purposes of the present application, vessel imaging techniques serve to provide diameter measurement values for the vessel lumen and are incorporated with blood viscosity to generate shear stress values. Using, for example, finite element analysis, the fluid dynamic properties of a patient's blood can be intertwined with patient-specific vasculature computationally, providing a diagnostic framework for assessing the risk of atherothrombosis. In such cases, threshold wall shear stress values can be identified in a patient specific manner. A clinical outcomes trial would be used to generate a risk ratio or score from the threshold wall shear stress values used in this diagnostic framework.

Friction here refers to a force that resists the motion of blood flow. Shear stress is tangential, frictional stress. Shear stress differs from circumferential tensile stress; while the former is dependent on viscosity, the latter is dependent on blood pressure. Frictional stress refers to a resisting force per unit area of blood vessel, and is usually reported using the unit dyne/cm². Shear stress is a function of shear rate (Shear Stress˜Shear Rate or τ≈\dot{γ}), with shear rate defined as the flow velocity over the diameter of the lumen of, for example, an artery. Measuring the shear stress caused by blood requires first measuring the viscosity of blood across a shear rate range because blood viscosity varies widely with shear rate, which is the ratio between the rate of flow of blood and the lumen diameter. Such blood viscosity measurements require multiple viscosity measurements; or a scanning-type viscometer; such as a dual riser/single capillary viscometer (U.S. Pat. No. 6,322,524); or a constitutive method for determining a viscosity-shear rate relationship (U.S. Pat. No. 6,796,168). Specifically, shear stress τ caused by a fluid is defined as viscosity multiplied by the shear rate \dot{γ} and expressed as:

$$τ = μ \dot{γ} = μ [ 8 \frac{V}{d} ] \qquad Formula 1$$

The proportionality μ that relates shear stress and shear rate is fluid viscosity, which can be understood as the inherent resistance of fluid to flow. V and d are the mean blood velocity and lumen diameter, respectively. Thus, if blood flow velocity and diameter of the vessel are fixed in a given vessel segment, the shear stress would depend primarily on the blood viscosity.

Wall shear stress in a human coronary can be estimated by applying Poiseuille's Law as follows:

$${τ}_{w} = μ [ \frac{8 V}{d} ] = \frac{4 Q μ}{π {r}^{3}} ( \frac{dyne}{{cm}^{2}} ) \qquad Formula 2$$

where τ_{W}=wall shear stress in dynes/cm², Q=coronary blood flow rate distal to the site of constriction in ml/s, μ=viscosity of the blood in units of poise, and r=the luminal radius at the site of constriction in centimeters.

In Formula 2, for simplicity sake, the wall shear stress is defined for a Newtonian fluid. Most common fluids such as water are Newtonian, which means that their viscosities do not vary over a range of shear rates. However, native blood is a non-Newtonian fluid meaning its viscosity varies over a range of shear rates (experienced physiologically). That is, blood is thicker when moving more slowly or through a wider vessel, and thinner when moving more quickly or through a more narrow vessel. Therefore, the numerical constant “8” (eight) used in Formula 2 will differ for a non-Newtonian fluid such as blood.

When the coronary artery does not have any plaque formation, pulsatile blood flow in a circular lumen geometry (e.g. the lumen of an artery) is not pathological. For example, the wall shear stress at the peak of systole (i.e. the highest shear rate) is in a range of approximately 10-15 dyne/cm². However, when relatively mild atherosclerotic plaque develops in the coronary artery, pathogenetic risk is expected to increase. Specifically, it is known that plaque rupture can lead to sudden heart attack (acute myocardial infarction); or if plaque rupture does not cause blood flow to be completely obstructed, the subject can experience intense chest pain (unstable angina); should a thrombus from a ruptured plaque migrate to the brain, a stroke (cerebrovascular accident); or to the lungs, a pulmonary embolism. However, the root cause of plaque rupture is simply not known and a subject of wide and intense debate. The inventor believes that the rupture of plaque is primarily due to increases in shear stress which can be caused by increases in blood viscosity and exacerbated by the blockage of the lumen of a blood vessel (e.g. an artery) by plaque formation. More specifically, it is asserted that the increase in blood viscosity is a central, overlooked factor responsible for elevating shear stress during a cardiac cycle to cause the rupture of plaque.

A cardiac cycle revolves from systole to diastole and back again. In a cardiac cycle, blood moves at a relatively high velocity during systole, while it moves more slowly during diastole. Blood actually stops moving for a brief moment during the transition from systole to diastole as the aortic valve closes inwardly toward the left ventricle.

Normally, blood viscosity is about 4 centiPoise [cP] during systole and increases to about 20 centiPoise [cP] during diastole. Thus, blood viscosity changes by a factor of five to ten within a single cardiac cycle, that is, a single heartbeat. Therefore, the changing viscosity of blood over a cardiac cycle adds an additional important dimension to predicting the risk for atherothrombosis and in particular to quantifying and predicting the likelihood of plaque rupture.

As outlined above, the viscosity of blood varies with its shear rate. According to one aspect of the present invention, the non-Newtonian characteristics of blood, and in particular, its changing viscosity within a single heartbeat, are taken into account to predict analytically the likelihood of atherothrombosis. Therefore, according to one aspect of the present invention, the effect caused by the viscosity of blood at high shear rate (systole) and the effect caused by the viscosity of blood at low shear fate (diastole) are analyzed separately.

In a method according to the first embodiment of the present invention, a systolic blood viscosity value (i.e. blood viscosity at a high shear rate) can be used to determine the propensity of atherosclerotic plaque to rupture. Any viscosity value measured at shear rate greater than 50 s⁻¹ may be used as a systolic blood viscosity value, however the inventor suggests that 300 s⁻¹ is an ideal shear rate for measuring systolic blood viscosity. It should be understood that 300 s⁻¹ may be one of many such acceptable shear rates. Beyond 300 s⁻¹, there is no change in the blood viscosity, and, therefore, blood viscosity at 300 s⁻¹ is an ideal measure of what viscosity would be at the highest shear rate. It is known that the viscosity of normal blood is about 4 cP at a shear rate of 300 s⁻¹ and does not thin further with increasing shear.

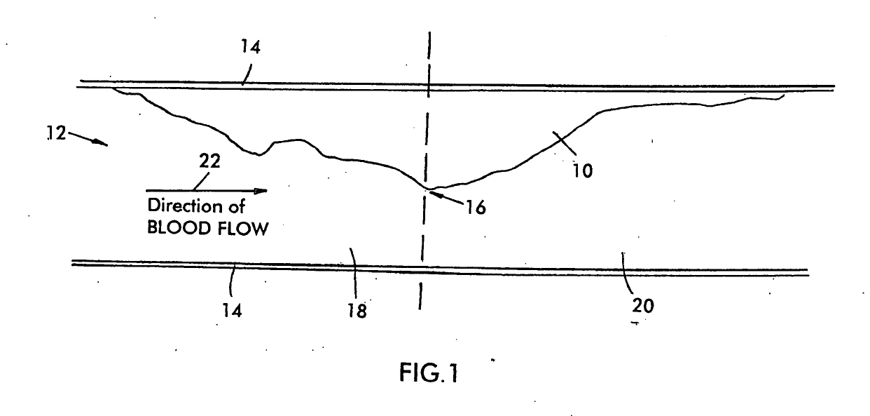

Referring now to FIG. 1, which illustrates a typical plaque formation 10 inside the lumen 12 of an artery 14, the increase in systolic blood viscosity increases the shear stress of blood which will have an adverse impact on the flow of blood at the stenosed artery, particularly at the proximal side (region upstream from the point of maximum blockage 16) 18 of the atherosclerotic plaque. Knowing the value of blood viscosity at high shear rate (systolic blood viscosity) for normal blood, and measuring the value of the blood viscosity of a patient at high shear rate can allow for calculating the increased risk of plaque rupture. Specifically, for example, a patient's blood viscosity at a high shear rate (e.g. 300 s⁻¹) which corresponds to the shear rate at the peak of systole can be measured and then compared to the viscosity of normal blood at the same shear rate. The difference between the two values can be then taken, divided by the viscosity of normal blood at the given shear rate and multiplied by 100 to obtain a percentage, which is indicative of the percentage of increase of the patient's blood viscosity compared to a person having normal blood. Because shear stress is a function of blood viscosity, shear stress can also be deemed to be elevated. The value so obtained can be compared to a threshold blood viscosity value to determine whether the plaque is at risk of rupture.

For example, assuming normal blood would have a systolic blood viscosity of 4 cP, and the patient's blood is measured to have a systolic blood viscosity of 5.6 cP, then the percentage increase in the blood viscosity of the patient over a normal value would be ((5.6−4)/4)*100=40%. Through a clinical outcome trial, the additional risk for atherothrombosis associated with such an elevated blood viscosity value can be determined and a threshold systolic blood viscosity value determined. If the threshold systolic blood viscosity value for plaque rupture has been reached (or surpassed), then it can be said that there is an increase in the risk of plaque rupture due to the increase in the viscosity of blood at systole. If not, it can be said that there is less risk of plaque rupture at systole. The risk ratio for an arterial thrombotic event can also be provided. Thus, a method according to the first embodiment provides a diagnostic framework for assessment of risk of plaque rupture based on a measurable, physical value, namely systolic blood viscosity.

As explained above, the viscosity of blood is elevated at lower shear rates. Therefore, during diastole, the viscosity of blood is increased. Elevated diastolic blood viscosity (elevated blood viscosity during diastole) results in an elevated diastolic shear stress, which the inventor believes, is a primary factor responsible for plaque growth, erosion, and rupture.

In a method according to the second embodiment of the present invention, a diastolic blood viscosity value (i.e. blood viscosity at a low shear rate) can be used to determine the propensity of atherosclerotic plaque to rupture. Any viscosity value measured at shear rate less than 25 s⁻¹ may be used as a diastolic blood viscosity value, however the inventor suggests that 1 s⁻¹ is an ideal shear rate for measuring diastolic blood viscosity. It should be understood that 1 s⁻¹ may be one of many such acceptable shear rates. Normal blood viscosity at a shear rate of 1 s⁻¹ is about 20 cP. In severe cases, diastolic blood viscosity can have a value of 100 to 200 percent greater than the diastolic blood viscosity of normal blood, serving as a reference value. For instance, a healthy subject can have a diastolic blood viscosity of 20 cP at shear rate of 1 s⁻¹, while in comparison, a patient with poor blood flow can have a diastolic blood viscosity measured at shear rate of 1 s⁻¹ of 60 cP.

Referring to FIG. 1, when diastolic blood viscosity is elevated, blood flow in the distal region (region downstream from the point of maximum blockage 16) 20 to a plaque is more turbulent. Increased turbulence expands the area of flow recirculation—the eddy—in the distal region 20. As the zone of flow recirculation widens along the flow direction 22 with the elevated diastolic blood viscosity, the arterial wall with dysfunctional endothelial cells expands downstream to the point of maximum blockage 16, accelerating plaque growth in the distal region 20.

In addition to the acceleration of plaque growth, elevated diastolic blood viscosity adversely affects existing plaque in the distal region 20, leading to the erosion of the plaque and the increased risk of acute coronary syndromes. Specifically, because of the increase in the blood viscosity during diastole, the diastolic shear stress is increased. The inventor suggests that this increase in shear stress may be a primary factor in the rupture of plaque. To quantify the increase in the risk of rupture, a calculation similar to the one used in the first embodiment can be used. Specifically, for example, a patient's diastolic blood viscosity can be measured and then compared to the viscosity of normal blood at the same shear rate. The difference between the two values can be then taken, divided by the viscosity of normal blood at the asymptotic diastolic shear rate and multiplied by 100 to obtain a percentage, which is indicative of the percentage of increase of the patient's blood viscosity compared to a person having normal blood. Because shear stress is a function of blood viscosity, shear stress can also be deemed to be elevated. The value so obtained can be compared to a threshold blood viscosity value to determine whether the plaque is at risk of rupture.

For example, assuming normal blood would have a diastolic blood viscosity of 20 cP, and the patient's blood is measured to have a diastolic blood viscosity of 60 cP, then the percentage increase in the blood viscosity of the patient over a normal value would be ((60−20)/20)*100=200%. Through a clinical outcome trial, the additional risk for atherothrombosis associated with such an elevated blood viscosity value can be determined and a threshold diastolic blood viscosity value determined. If the threshold diastolic blood viscosity value for plaque rupture has been reached (or surpassed), then it can be said that there is an increase in the risk of plaque rupture due to the increase in the viscosity of blood at diastole. If not, it can be said that there is less risk of plaque rupture at diastole. The risk ratio for an arterial thrombotic event can also be provided. Thus, a method according to the second embodiment provides a diagnostic framework for assessment of risk of plaque rupture based on a measurable, physical value, namely diastolic blood viscosity.

As is known, plaque formation reduces the diameter of the lumen of a vessel, such as an artery. The reduction in the diameter of the lumen increases shear stress as can be discerned from Formula 1 and Formula 2. In a method according to the third embodiment of the present invention the change in blood viscosity can be used in combination with values indicative of the magnitude of the blockage (e.g. expressed as percent of blockage) of the lumen to assess the risk for the rupture of plaque.

A certain percent blockage of an artery by plaque is considered by medical practitioners to be asymptomatic and non-pathological. For example, 30% blockage of an artery by plaque is considered asymptomatic and non-pathological. Note, however, that acute cardiovascular events such as heart attacks occur in patients with little or no symptoms as well as those without any conventional risk factors (e.g., high blood pressure, elevated cholesterol levels, etc.) It is an aspect of the third embodiment of this invention to measure and monitor increases in blood viscosity at a high shear rate (systolic blood viscosity) combined with the reduction in the lumen due to blockage, specifically for the purpose of measuring changes in systolic shear stress, which can result in the rupture of plaque even when the blockage is at a safe level for a person—whether or not the patient's systolic blood viscosity alone is indicative of increased risk.

The luminal radius of a stenosed coronary artery at the site of diameter reduction can vary such that the percentage blockage varies from zero to 90% or more. Also, systolic blood viscosity, that is, at high shear rates can vary from 3.5 to 6.0 cP in patients with no other symptoms or risk factors. Assuming that coronary blood flow in the left anterior descending coronary artery distal to the site of subcritical stenosis can be estimated as 150 ml/min with maintenance of normal arterial pressure at rest, during exercise, coronary blood flow can increase three-fold to 450 ml/min. Because shear stress is a function of blood flow rate (see Formula 2), in the outlined example, the increase in the blood flow during exercise would increase the wall shear stress three-fold. Thus, if one has elevated systolic blood viscosity, there will be additional increase in the wall shear stress, increased frictional force, and increased tendency for plaque rupture and atherothrombosis due to the blockage as well as increased blood flow.

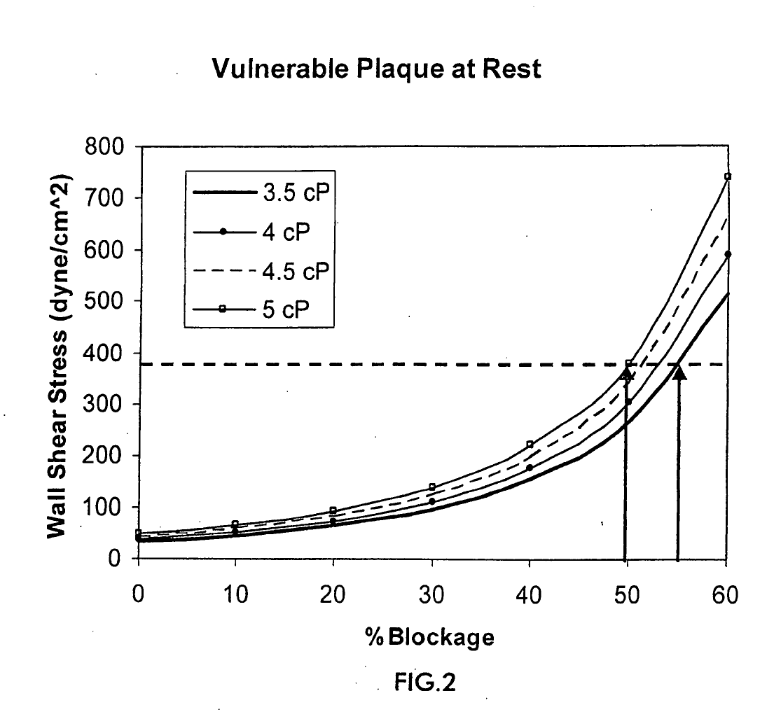

Referring to FIG. 2, assuming for illustration purposes only that the threshold shear stress value for rupturing plaque is 380 dyne/cm² (this value should not be understood to be a statistically significant threshold wall shear stress value as determined through clinical trials and is only being used to illustrate the concept underlying the third embodiment of the present invention). A set of wall shear stress values can be calculated for a number of systolic blood viscosity values as a function of the percentage blockage of an artery while the patient is at rest (i.e. at normal coronary flow rate). Note that at a systolic blood viscosity of 3.5 cP (see bottom curve), the calculated wall shear stress reaches 380 dyne/cm² at 55% blockage, whereas at a systolic blood viscosity of 5.0 cP (see top curve), the calculated wall shear stress reaches 380 dyne/cm² at 50% blockage. Thus, FIG. 2 indicates that there can be plaque rupture at a lower blockage percentage if the blood viscosity of the patient is elevated.

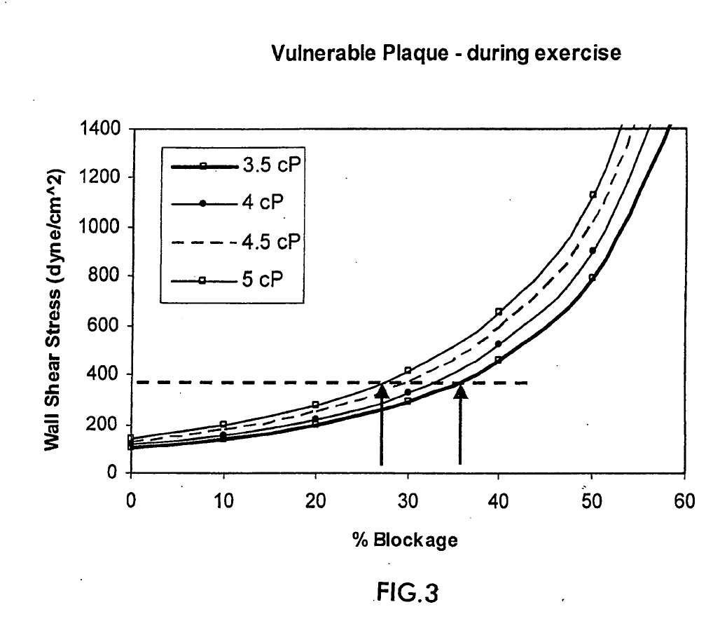

FIG. 3 shows wall shear stress values for a number of systolic blood viscosity values as a function of percent blockage of the artery during exercise when the coronary blood flow is assumed to increase three-fold to, for example, 450 ml/min (i.e. high blood flow condition). FIG. 3 shows that at a blood viscosity value of 3.5 cP (see bottom curve), the calculated wall shear stress reaches 380 dyne/cm² at 36% blockage, whereas at a blood viscosity of 5.0 cP (see top curve), the calculated wall shear stress reaches 380 dyne/cm² at 27% blockage. Thus, the increased blood flow combined with the increase in the systolic blood viscosity can result in a elevated wall shear stress even at a lower percentage of blockage.

FIGS. 2 and 3 illustrate that percent blockage of an artery can be used in conjunction with systolic blood viscosity values (measured, for example, at a systolic shear rate such as 300 s⁻¹) to determine whether there is a risk of plaque rupture. Thus, systolic blood viscosity as well as the percent blockage can be used to calculate shear stress using, for example, Formulas 1 and 2. Through a clinical outcome trial, the risk ratios associated with such an elevated systolic shear stress can be determined and a threshold systolic shear stress value determined. If the threshold systolic shear stress value for plaque rupture has been reached, then it can be said that there is an increase in the risk of plaque rupture due to the increase in the shear stress at systole. If not, it can be said that there is less risk of plaque rupture at systole. The risk ratio for an arterial thrombotic event can also be provided. The calculated shear stress can be then compared to a threshold shear stress value to determine whether the plaque is at risk of rupture. Thus, for example, if the calculated shear stress is above the threshold shear stress value, it can be said that there is higher risk for plaque rupture. However, if the calculated shear stress value is less than the threshold shear stress value, it can be said that there is a lower risk of rupture.

FIG. 3 illustrates that increased blood flow can heighten the risk of plaque rupture by increasing shear stress. This example illustrates a diagnostic framework that provides utility for an otherwise healthy patient (e.g., a 40 year old male, who has none of the conventional cardiovascular risk factors and maintains a healthy lifestyle in terms of diet and exercise), whose systolic blood viscosity is elevated. Thus, in addition to systolic blood viscosity and percent blockage, using Formulas 1 and 2, for example, shear stress under high blood flow conditions—i.e., with intense exercise or stress—can be calculated and compared with the threshold shear stress value to determine whether there is risk for rupture of plaque. Through a clinical outcome trial, the risk ratios associated with such an elevated systolic shear stress can be determined for subjects under high flow conditions (i.e., high cardiac output as with exercise) and a threshold systolic shear stress value determined. Cardiac output, lumen diameter, viscosity, and shear stress can all be measured clinically and associated with increased risk of arterial thrombotic events to create a risk profile and threshold values. For example, if the calculated shear stress is above the threshold shear stress value, it can be said that there is higher risk for plaque rupture. However, if the calculated shear stress value is less than the threshold shear stress value, it can be said that there is a lower risk of rupture. Thus, in a method according to the third embodiment, the risk of plaque rupture in view of percentage blockage of the artery as well as increased cardiac output can be predicted using systolic blood viscosity.

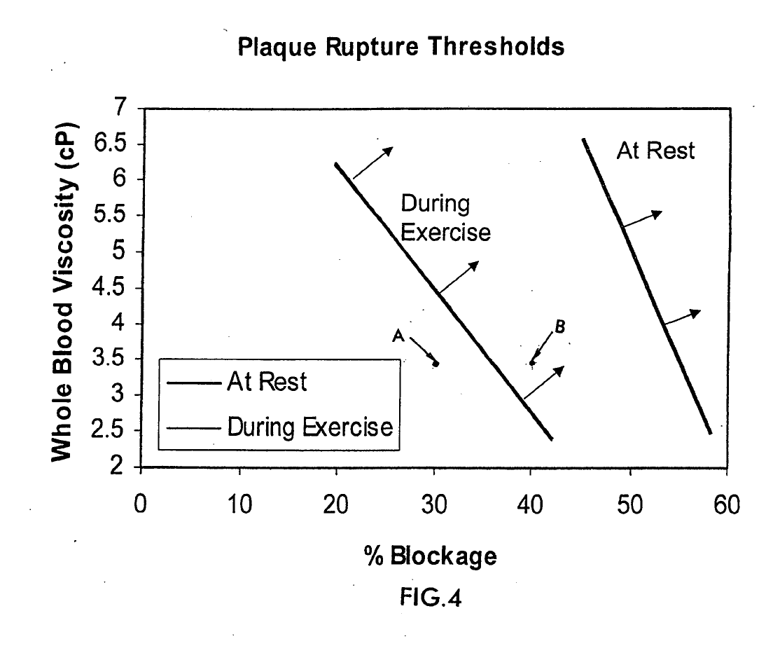

Referring to FIG. 4 assuming, for example, that the threshold systolic shear stress value is 380 dyne/cm², a value for systolic blood viscosity that would result in a shear stress value of 380 dyne/cm² for a given percent blockage can be calculated for blood flow rate at rest and during elevated blood flow (e.g. exercise). Each value so calculated can be plotted to obtain curves relating systolic blood viscosity to percent blockage for ease of analysis. The zone to the right of each curve would indicate the zone of vulnerable plaque—as determined uniquely through blood flow parameters, and not by plaque morphology as commonly done. Thus, for example, knowing a patient's systolic blood viscosity, which can be measured, and the percentage blockage of an artery, which can also be determined, it can be determined whether a patient is at risk of atherothrombosis if he or she should safely engage in exercise and if so what kind of exercise. For example, a patient whose blood has a systolic blood viscosity of 3.5 cP, and whose artery is 30% blocked, should not suffer from plaque rupture even during exercise (See Point A). On the other hand, a patient having the same systolic blood viscosity, but 40% (see point B) blockage should be advised of an increased likelihood of plaque rupture during exercise. Specific threshold values can be obtained through clinical outcome trials.

In a method according to the fourth embodiment of the present invention, in addition to systolic blood viscosity, and percent blockage of the lumen, local surface irregularities of the plaque formation can be taken into account to calculate high shear stress locations.

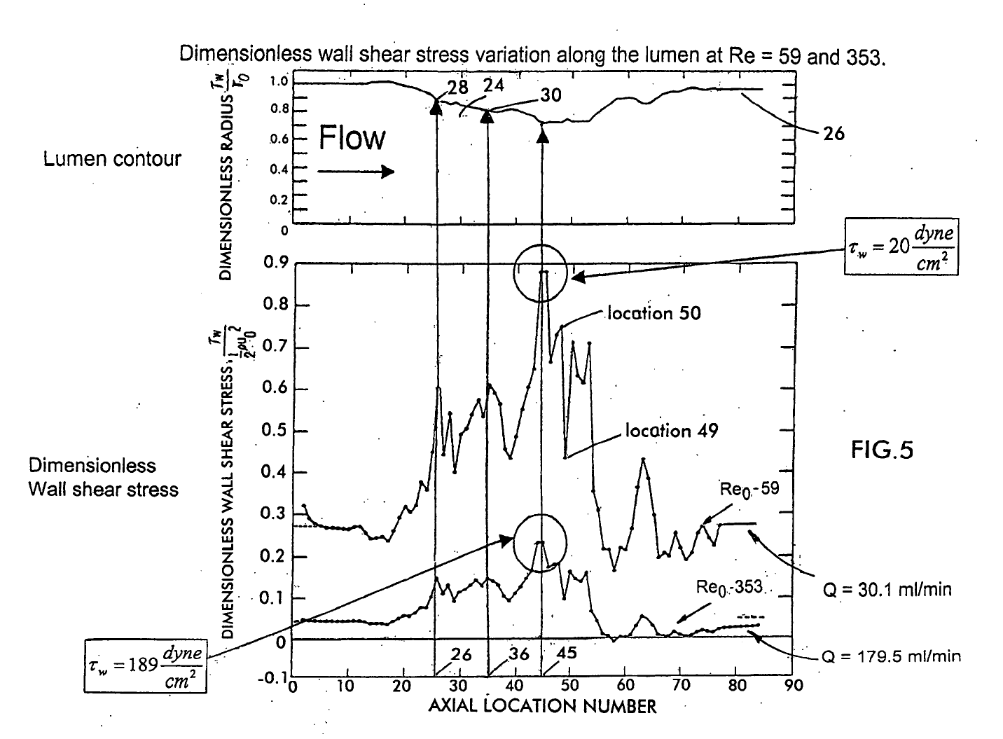

FIG. 5 illustrates contours of a plaque formation normalized by inlet radius to accentuate local wall irregularities, which was the subject of L. H. Back et al. [35]. FIG. 5 further provides published predictions of the distributions of wall shear stress (normalized by the dynamic pressure at the inlet) at Reynolds numbers of 59 and 353 [35]. Note that Reynolds numbers are used as units to quantify flow turbulence in the lumen. Back et al. [35] performed this study assuming blood is Newtonian (having a viscosity of 4 cP) and focused exclusively on conditions at the peak of systole.

Generally, FIG. 5 shows increases in the wall shear stress in the contraction region (region upstream from the point of highest percent blockage) 24 to peak values at the narrowest location (the point of highest percent blockage) indicated by Axial Location No. 45, and subsequent decreases in the divergent region (region downstream from the point of highest percent blockage) 26 downstream of the maximum constriction region (axial location numbers above 45). FIG. 5 clearly shows sharp variations in the wall shear stress that correspond with local wall irregularities; with sharp and abrupt changes. For example, in the contraction region 24 local increases in the wall shear stress at locations No. 26 and No. 36 can be observed in the vicinity of local lesions 28, 30, respectively. However, in the trough regions just downstream of these lesions, there were local decreases in the wall shear stress. The largest local changes in shear stress occurred in the narrowest region from locations No. 45 to No. 50 where relatively small wall irregularities produced drastic variations. Specifically, from location No. 45 to No. 49 the wall shear stress decreased by about 50%, then increased again at location No. 50 by about 65% above that at location No. 49.

FIG. 5 indicates that wall shear stress can be affected dramatically by localized changes in the surface of the plaque formation.

Based on the findings of FIG. 5, it can be concluded that it is possible to calculate the wall shear stress at the contraction region of the lumen using a computational fluid dynamic method based, for example, on the Navier-Stokes equation [36] and accurate lumen diameter data in the stenosed artery along the flow direction. As a result, wall shear stress at each axial location along the direction of flow of blood can be calculated, whereby, for example, changes in the shear stress due to local abrupt changes can be identified.

Thus, in a method according to the fourth embodiment of the present invention, first a profile of the plaque formation is obtained, and then using the profile so obtained the shear stress on the surface of the plaque formation is calculated along the axial direction of the flow, whereby regions of high shear stress can be identified. More specifically, the shear stress at each location is calculated using the diameter of the lumen at that location, and then the shear stress so calculated is compared to a threshold shear stress to determine whether any part of the plaque formation is at a risk of rupture as explained above. Note that in addition to taking into account the change in the local diameter of the lumen to calculate shear stress at that location, the viscosity of the blood is also taken into account. Thus, in the contraction region 24, i.e. the region up stream from the point of maximum blockage, the systolic blood viscosity may be used, while in the divergent region 26, i.e. the region down stream from the point of maximum blockage, the diastolic blood viscosity can be used to determine the shear stress.

In order to obtain the blood vessel diameter data, one can use various blood vessel imaging methods including angiography, interferometric phase-contrast imaging technique, magnetic resonance imaging (MRI), three-dimensional MR angiography, CT, intravascular ultrasound, virtual arterial endoscopy, and endovascular probe, among others. Blood flow velocities can be measured using a wide variety of techniques including ultrasonic, radiographic, electromagnetic, pressure transducing, anemometric methods, among others, both extracorporeal and percutanous, which can assist in the selection of shear rates used for viscosity measurement. In a method according to the fourth embodiment, an image of the stenosed portion of the artery can be obtained, the image is then analyzed to obtain a value for the diameter of the lumen for a number of locations along the flow direction in the stenosed portion of the artery, and a diameter value at each location can be used to determine the wall shear stress at that location. Flow velocity information is also obtained. Additionally, blood viscosity values are obtained for shear rates relevant to the clinical application (or ideally for a comprehensive range of shear rates). Each shear stress value can be then compared to a threshold critical wall shear stress value to determine whether the plaque at that location is at a risk of rupture.

In the divergent region where the predicted wall shear stress decreases, the level of wall shear stress was closer to the upstream values (i.e. dashed line in FIG. 5) at the lower Reynolds number of 59 than at the higher Reynolds number of 353. The lower Reynolds number of 59 can be understood to correspond to normal cardiac output, and the high Reynolds number of 353 can be understood to correspond to elevated cardiac output, as in the case of intense exercise. Note, the shear stress values in FIG. 5 are dimensionless, so the values on the y-axis denote relative changes rather than absolute value.

The role of the local lesion centered at location No. 63 plays an important part in establishing the level of wall shear stress in the divergent region. As observed in FIG. 5, there is a sharp decrease in wall shear stress just downstream of the narrowest region at location No. 53. While location No. 53 experiences systolic shear stress, location No. 63 experiences diastolic shear stress. In the region under diastolic shear stress such as at location No. 63, endothelial cells become dysfunctional, meaning that the endothelial cells take on a rounded shape instead of normal elliptic shape. The dysfunctional endothelial cells produce leaky sites, with increased permeability of the endothelial layer at the low shear rate or diastolic flow regime. Oxidized LDL molecules can then easily penetrate from the blood stream to the arterial wall, an event that is important to the pathophysiological process of atherosclerosis. This underscores the utility of combining diameter information with viscosity to determine the impact of plaque formation on the friction caused by blood flow: in the case of location No. 63, diastolic shear stress; in the case of location No. 53, systolic shear stress.

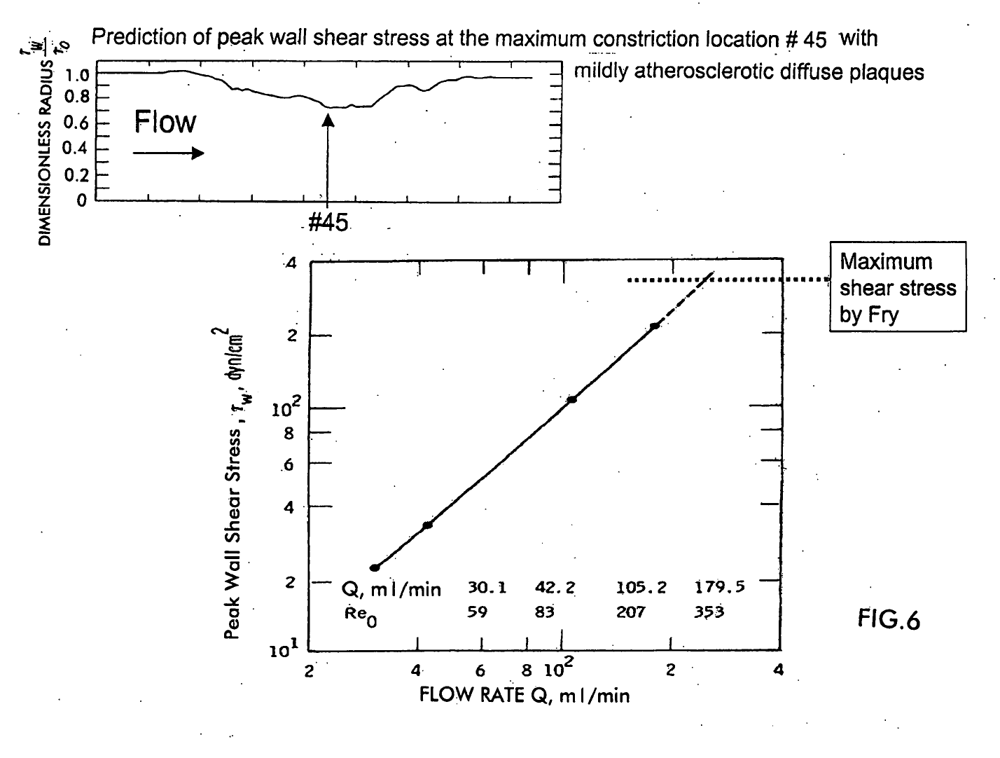

FIG. 6 shows a plot of peak wall shear stress values at the narrowest location in FIG. 5 (i.e. highest percent blockage), No. 45, as a function of the flow rate of blood for a number of Reynolds numbers [35]. FIG. 6 shows that the highest predicted wall shear stress at a Reynolds number of 353 was 215 dyne/cm², while the estimated peak shear stress at a Reynolds number of 500 is about 350 dyne/cm². Thus, by taking account of the percent blockage, the flow rate of blood, and the Reynolds number, a peak shear stress can be calculated, which can be then compared to a threshold shear stress value to determine whether the plaque at the location under analysis (in the case of FIG. 6 the location of the highest percent of blockage) is at risk of rupture.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. A method for determining risk of rupture of a portion of a plaque formation residing in a lumen of a human blood vessel, comprising:
calculating a reference blood viscosity value based on a reference blood shear rate value;
calculating a reference blood shear stress value based on said reference blood viscosity value;
comparing said reference blood shear stress value to a critical threshold shear stress value indicative of a critical shear stress required to rupture plaque to determine whether said blood shear stress value has at least reached said critical shear stress value.

2. The method of claim 1, wherein said reference blood shear rate value is higher than 50 s⁻¹.

3. The method of claim 1, wherein said reference blood shear rate value is higher than 300 s⁻¹.

4. The method of claim 1, wherein said reference blood shear rate value is less than 25 s⁻¹.

5. The method of claim 1, wherein said reference blood shear rate value is less than 1 s⁻¹.

6. The method of claim 1, wherein said reference blood shear stress value is calculated based on said reference blood viscosity value and percent blockage of said lumen by said plaque formation.

7. The method of claim 1, wherein said reference blood shear stress value is calculated based on said reference blood viscosity value, said percent blockage of said lumen, and a blood flow velocity value.

8. The method of claim 7, wherein said blood flow velocity value is based on increased cardiac output due to elevated heart rate and exercise.

9. The method of claim 1, wherein said reference blood shear stress value is calculated based on said reference blood viscosity value and diameter value of said lumen at a location corresponding to a location on said plaque formation.

10. The method of claim 9, wherein said diameter value is obtained through at least one method selected from a list consisting of angiography, interferometric phase-contrast imaging, magnetic resonance imaging, three-dimensional MR angiography, computed tomography, intravascular ultrasound, virtual arterial endoscopy, and endovascular probe.

11. The method of claim 9, wherein said reference blood shear rate value is higher than 50 s⁻¹.

12. The method of claim 9, wherein said reference blood shear rate value is higher than 300 s⁻¹.

13. The method of claim 9, wherein said reference blood shear rate value is less than 25 s⁻¹.

14. The method of claim 9, wherein said reference blood shear rate value is less than 1 s⁻¹.

15. The method of claim 1, further comprising determining a risk ratio based on the result of said comparing step.
